(19) United States
(12) Patent Application Publication
(10) Pub. No.: US 2026/0266778 A1
(43) Pub. Date: Sep. 10, 2026

(54) METHOD FOR ANALYZING DEGRADATION OF MARINE BIODEGRADABLE PLASTIC

(71) Applicant: SHIMADZU CORPORATION, Kyoto-shi, Kyoto (JP)

(72) Inventors: Chihiro NOUDA, Kyoto-shi, Kyoto (JP); Kana KUROISHI, Kyoto-shi, Kyoto (JP); Makoto YASOJIMA, Kyoto-shi, Kyoto (JP); Hiroaki TAKEMORI, Kyoto-shi, Kyoto (JP)

(73) Assignee: SHIMADZU CORPORATION, Kyoto-shi, Kyoto (JP)

(21) Appl. No.: 19/162,919

(22) PCT Filed: Apr. 26, 2024
(86) PCT No.: PCT/JP2024/016374

§ 371 Date: Sep. 5, 2025

(30) Foreign Application Priority Data
Mar. 15, 2023 (JP) 2023-040548

(51) Int. Cl. G01N 30/72; G01N 30/02; G01N 30/14; G01N 30/88

(52) U.S. Cl. CPC G01N 30/7233; G01N 30/14; G01N 30/88; G01N 2030/027; G01N 2030/885

(57) ABSTRACT

To provide an analysis method that can provide information on the degradation mechanism of marine biodegradable plastics. This analysis method is a method for analyzing degraded intermediates of a marine biodegradable plastic, comprising an extraction step of extracting pore water present in a biofilm formed on a surface of the marine biodegradable plastic, and a detection step of detecting the degraded intermediates in the pore water using mass spectrometry.

### TECHNICAL FIELD

The present invention relates to a method for analyzing degraded intermediates of a marine biodegradable plastic.

### BACKGROUND ART

In response to the problem of marine plastic waste, the demand for marine biodegradable plastics that are degraded by microorganisms in the ocean is increasing, and product development is advancing. Specifically, a biodegradable polyester composed of a polymer of 3-hydroxybutyric acid and 3-hydroxyhexanoic acid has been proposed and is commercially available (see Patent Literature 1).

For such marine biodegradable plastics, the elucidation of the biodegradation mechanism, which is information necessary for product development according to the application, has not progressed. With conventional methods, the biodegradation of marine biodegradable plastics is evaluated by methods such as quantifying the amount of gas consumed as degradation progresses (ISO 23977-2), or comparing the weight of the plastic before and after biodegradation (ISO 22766).

### CITATION LIST

### Patent Literature

[Patent Literature 1] JP 2007-259708 A

### SUMMARY OF INVENTION

### Technical Problem

However, since all conventional methods are indirect, it is not possible to ascertain what kind of degraded intermediates are generated and distributed during the degradation process. Grasping such information on degraded intermediates is helpful for elucidating the degradation mechanism, and therefore the development of a new analysis method is required.

An object of the present invention is to provide an analysis method that can provide information on the degradation mechanism of marine biodegradable plastics.

### Solution to Problem

An analysis method according to a first aspect of the present invention is a method for analyzing degraded intermediates of a marine biodegradable plastic, comprising an extraction step of extracting pore water present in a biofilm formed on a surface of the marine biodegradable plastic, and a detection step of detecting the degraded intermediates in the pore water using mass spectrometry.

### Advantageous Effects of Invention

According to the analysis method of the first aspect, it is possible to ascertain the presence/absence and degree of polymerization of degraded intermediates generated during the degradation of a marine biodegradable plastic, which can be useful for elucidating the degradation mechanism.

### BRIEF DESCRIPTION OF DRAWINGS

FIG. 1 shows an image of one marine biodegradable plastic pellet with an attached biofilm prepared in Example 1.

FIG. 2 shows the concentration of degraded intermediates extracted and detected in Example 1, where the left graph shows a concentration range of 0-500 μg/ml, and the right graph is a partial enlarged view of the left graph, showing a concentration range of 0-5 μg/ml. In the graphs, the vertical axis indicates the concentration in the pore water, and the horizontal axis indicates the number of biodegradation days.

### DESCRIPTION OF EMBODIMENTS

### 1. First Embodiment

The analysis method of the first embodiment of the present invention is a method for analyzing the degradation of a marine biodegradable plastic, specifically, its degraded intermediates, and comprises a preparation step, an extraction step, and a detection step in order. Each step will be described in detail below.

### (Preparation Step)

In this step, a marine biodegradable plastic with a biofilm attached to its surface is prepared as an analysis sample.

The marine biodegradable plastic may be any polymer that degrades in seawater, and examples include polyhydroxyalkanoates. Examples of monomers constituting the polyhydroxyalkanoate include 3-hydroxybutyric acid, 3-hydroxyisopentanoic acid, 3-hydroxyhexanoic acid, 3-hydroxyoctanoic acid, 3-hydroxydecanoic acid, 3-hydroxyundecanoic acid, 3-hydroxytetradecanoic acid, and 3-hydroxyhexadecanoic acid. In the first embodiment, a representative example of an analysis sample of a marine biodegradable plastic is a polymer having the structure represented by the following Chemical Formula (1), that is, a copolymer of 3-hydroxybutyric acid and 3-hydroxyhexanoic acid. In the formula, x is an integer of 1 or more, and y is an integer of 1 or more.

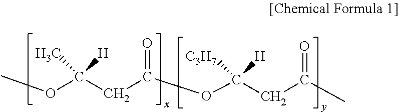

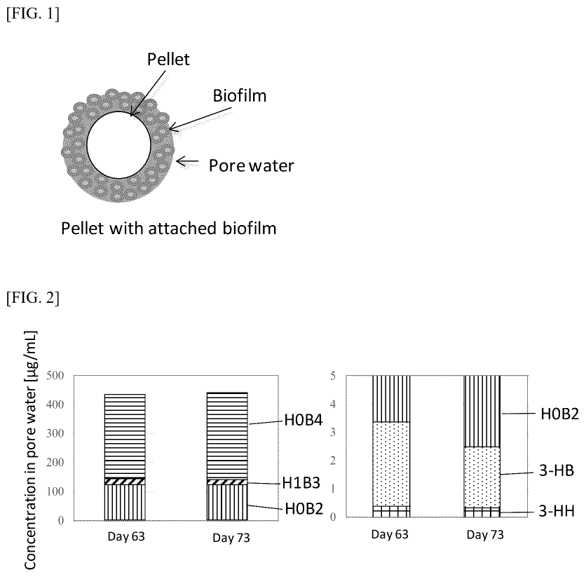

The marine biodegradable plastic with a biofilm formed on its surface (hereinafter referred to as biofilm-attached plastic) is obtained when the marine biodegradable plastic is in contact with seawater for a certain period or longer. Specifically, it may be a marine biodegradable plastic that has been floating or settled in the sea for a certain period of time; or a marine biodegradable plastic that has been in contact with natural or artificial seawater for a certain period of time (that is, a marine plastic subjected to a biodegradation test). Generally, a marine biodegradable plastic is degraded by microorganisms in seawater, and at the same time, a biofilm, which is an aggregate of these microorganisms, is formed on its surface. Furthermore, as shown in FIG. 1, water is contained in the biofilm, which is an aggregate of microorganisms. As various microorganisms gather to form a biofilm, degrading enzymes released by the microorganisms hydrolyze the molecular chains of the polymer from the polymer surface, and the liberated oligomers are absorbed, assimilated, and mineralized by the microorganisms. Hereinafter, the water contained in the biofilm, which is an aggregate of microorganisms, is referred to as pore water.

### (Extraction Step)

In this step, the pore water present in the biofilm-attached plastic is extracted.

Examples of the extraction treatment include filtration (microfiltration, ultrafiltration, etc.), centrifugation, and a combination thereof. In the first embodiment, filtration is preferable from the viewpoint of reliably extracting the pore water, and a combination of filtration and centrifugation is particularly preferable. That is, the biofilm-attached plastic is subjected to centrifugation in a centrifugal filter unit with a membrane filter, and the liquid is passed through the membrane filter to extract a separated liquid, that is, the pore water.

The pore water extracted in this step contains degraded intermediates. The degraded intermediates are monomers and/or oligomers liberated by hydrolysis of the molecular chains of the polymer, which is the marine biodegradable plastic, by degrading enzymes produced by microorganisms present in seawater, and are of the monomer and/or oligomer units constituting the marine biodegradable plastic, and particularly, both the monomers and oligomers constituting the marine biodegradable plastic.

For example, when the marine biodegradable plastic is a polymer having the structure represented by Chemical Formula (1), the monomers are 3-hydroxybutyric acid and 3-hydroxyhexanoic acid. The oligomer is an oligomer composed of at least one of these monomers, for example, represented by the following Chemical Formula (2). In the formula, x is, for example, an integer of 1 or more and 15 or less, and y is, for example, an integer of 1 or more and 15 or less. The total degree of polymerization of the detected oligomer, that is, the sum of x and y, depends on the type of mass spectrometer, but when a liquid chromatograph-tandem mass spectrometer is used as the mass spectrometer, it is, for example, 20 or less, preferably 12 or less, more preferably 6 or less, still more preferably 4 or less, and is, for example, 1 or more. In particular, it is preferable to detect oligomers with a total degree of polymerization of 1 to 20, preferably 1 to 4 and 6, by time-of-flight mass spectrometry, and to perform quantitative analysis of oligomers with a total degree of polymerization of 1, 2, and 4 by tandem mass spectrometry (for example, triple quadrupole).

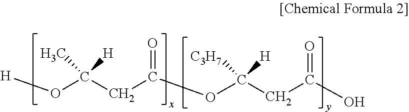

### (Detection Step)

In this step, mass spectrometry is performed using the pore water. Thereby, the degraded intermediates of the marine biodegradable plastic are detected.

Preferably, liquid chromatography-mass spectrometry is performed as the mass spectrometry method. That is, after the pore water is subjected to component separation by liquid chromatography, mass spectrometry is performed.

In liquid chromatography, any known or conventional column can be appropriately selected as long as it can retain the monomers and oligomers constituting the marine biodegradable plastic. For example, a column packed with octadecyl group-containing silica gel as the stationary phase can be mentioned. The mobile phase can be appropriately selected according to the column and the marine biodegradable plastic, and examples include acidic aqueous solutions such as formic acid aqueous solution, acetic acid aqueous solution, and phosphoric acid aqueous solution; and organic solvents such as methanol, ethanol, acetonitrile, and hexane. A mixed solvent of an acidic aqueous solution and an organic solvent is preferable.

In the first embodiment, a gradient elution method is preferably adopted. That is, the concentration of the organic solvent in the mobile phase is gradually increased from the initial stage of the analysis. This allows for the efficient separation of multiple types of degraded intermediates having a wide molecular weight distribution, and can shorten the run time.

In mass spectrometry, examples of ionization means include Electron Ionization (EI), Electrospray Ionization (ESI), and Atmospheric Pressure Chemical Ionization (APCI).

As for the type of mass separation, that is, the type of mass spectrometer, examples include a quadrupole mass spectrometer, a magnetic sector mass spectrometer, a time-of-flight mass spectrometer, an ion trap (including Orbitrap) mass spectrometer, and an ion cyclotron resonance mass spectrometer. A tandem mass spectrometer composed of multiple analyzers may also be used. That is, in the first embodiment, a liquid chromatograph-tandem mass spectrometer (LC/MS/MS) may be adopted. Examples of the type of tandem mass spectrometry (MS/MS) include a triple quadrupole (Q-Q) mass spectrometer, a tandem time-of-flight (TOF-TOF) mass spectrometer, a quadrupole-time-of-flight (Q-TOF) mass spectrometer, a quadrupole-ion trap (Q-IT) mass spectrometer, a quadrupole-ion cyclotron resonance (Q-ICR) mass spectrometer, and an ion trap-time-of-flight (IT-TOF) mass spectrometer.

From the viewpoint of enabling measurement of exact mass and facilitating accurate distinction and identification of chemical formulas between similar degraded intermediates, it is preferable to use an ion trap mass spectrometer, a time-of-flight mass spectrometer, or an ion cyclotron resonance mass spectrometer, particularly a time-of-flight mass spectrometer. From the viewpoint of facilitating accurate quantification of each degradation intermediate product, it is preferable to use a quadrupole mass spectrometer, particularly a triple quadrupole mass spectrometer. In the first embodiment, it is preferable to perform at least two types of mass spectrometry. Specifically, mass spectrometry using an ion trap mass spectrometer, a time-of-flight mass spectrometer, or an ion cyclotron resonance mass spectrometer, and mass spectrometry using a quadrupole mass spectrometer are performed. This makes it possible to identify the monomers and oligomers that are the degraded intermediates and to accurately measure their concentrations, thereby enabling more detailed information on the degradation mechanism to be obtained.

A conventional or commercially available liquid chromatograph mass spectrometer may be used for the liquid chromatography-mass spectrometry. Various setting conditions for the liquid chromatograph and the mass spectrometer may be appropriately set according to the recommendations for each device, with reference to the composition of the monomers and oligomers in the degraded intermediates.

Thereby, the degraded intermediates are detected. That is, a mass spectrum is obtained for each degradation intermediate product separated by liquid chromatography. In the mass spectrum, the m/z and peak intensity of ions (precursor ions, fragment ions, or product ions) derived from the monomers and/or oligomers constituting the degradation intermediate product are output. Then, in each of these mass spectra, each monomer and each oligomer constituting the degradation intermediate product can be identified based on the m/z and peak intensity of the various ions. For the calculation of their concentration (content), for example, a calibration curve showing the relationship between concentration and peak intensity may be prepared in advance by performing mass spectrometry on a degradation intermediate product of known concentration at multiple concentrations, and this calibration curve may be used for collation.

In this step, the degree of polymerization of the degraded intermediates, that is, various oligomers, can also be determined. That is, the m/z of these oligomers is calculated and associated in advance based on the structure and molecular weight of the degraded intermediates, that is, various oligomers, and the structure and molecular weight of the monomers constituting the oligomers. Then, by detecting the peaks of ions derived from the monomers and/or oligomers constituting the degraded intermediates in the mass spectrum obtained in the detection step and collating them with the corresponding m/z, the degree of polymerization of each oligomer can be determined.

According to the analysis method of the first embodiment, the degraded intermediates of a marine biodegradable plastic can be analyzed. It has been conventionally believed that when a marine biodegradable plastic degrades in seawater, its degradation products are immediately assimilated or mineralized, or do not exist at a detectable concentration level due to dilution by seawater. In contrast, the present inventors focused on the biofilm attached to the marine biodegradable plastic, predicted that degraded intermediates might exist at a measurable concentration level in the water contained in the biofilm, which is the very site where biodegradation is progressing, and actually confirmed that the pore water contained in the biofilm contains a large amount of degraded intermediates by measuring it with a mass spectrometer, leading to the invention of the first embodiment.

In particular, according to the analysis method of the first embodiment, it is possible to ascertain the presence/absence and degree of polymerization of degraded intermediates generated during the degradation of a marine biodegradable plastic, which can be useful for elucidating the degradation mechanism. Furthermore, by using this analysis method in parallel with conventional biodegradation test, it becomes possible to consider the relationship between the degree of biodegradation and degraded intermediates, which may lead to the proposal of a new biodegradation degree evaluation method.

### 2. Aspects

It will be understood by those skilled in the art that the exemplary embodiments described above are specific examples of the following aspects.

(Item 1) An analysis method according to one aspect may be a method for analyzing degraded intermediates of a marine biodegradable plastic, comprising an extraction step of extracting pore water present in a biofilm formed on a surface of the marine biodegradable plastic, and a detection step of detecting the degraded intermediates in the pore water using mass spectrometry.

(Item 2) In the analysis method according to item 1, the concentration of the degraded intermediates may be detected in the detection step.

(Item 3) In the analysis method according to item 1 or 2, the degraded intermediates may be a monomer and/or an oligomer constituting the marine biodegradable plastic.

(Item 4) In the analysis method according to item 3, the degraded intermediates may be a monomer and an oligomer constituting the marine biodegradable plastic.

(Item 5) In the analysis method according to item 3 or 4, the oligomer may be an oligomer with a total degree of polymerization of up to 20.

(Item 6) In the analysis method according to item 3 or 4, the oligomer may be an oligomer with a total degree of polymerization of up to 6.

(Item 7) In the analysis method according to item 3 or 4, the oligomer may be an oligomer with a total degree of polymerization of up to 4.

(Item 8) In the analysis method according to any one of items 1 to 7, the pore water may be extracted by filtering the biofilm formed on the surface of the marine biodegradable plastic.

(Item 9) In the analysis method according to any one of items 1 to 8, the mass spectrometry may use a time-of-flight mass spectrometer.

(Item 10) In the analysis method according to any one of items 1 to 9, in the detection step, after separating components of a solution in which the degraded intermediates have been extracted by liquid chromatography, mass spectrometry may be performed.

(Item 11) In the analysis method according to item 10, the liquid chromatography may be performed by gradient elution.

(Item 12) In the analysis method according to any one of items 1 to 11, the marine biodegradable plastic may have the structure represented by the above Chemical Formula (1).

### EXAMPLES

Next, the present invention will be described in detail with reference to Examples and Comparative Examples, but the scope of the present invention is not limited by these.

### Example 1

Ammonium chloride (19.1 mg/L) and disodium hydrogen phosphate (2.3 mg/L) were added to seawater collected in Osaka Bay to prepare test seawater, and 300 mL of this was poured into each of 20 500-mL glass test bottles. Twenty marine biodegradable plastic pellets, Kaneka biodegradable biopolymer Green Planet™ (registered trademark) (40 mg) represented by the chemical formula below, were prepared, and one pellet was immersed in each of the aforementioned test bottles. These were maintained at a water temperature of 27° C. while stirring the test seawater for a prescribed period (63 days, 73 days). During this time, the formation of a biofilm on the surface of the plastic pellets was visually confirmed (see FIG. 1).

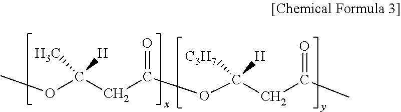

After each prescribed period had elapsed, the plastic pellets with the attached biofilm were taken out, placed in a centrifugal filter tube (pore size 0.2 μm), and centrifuged under the conditions of 1500 g for 15 minutes to collect pore water (separated liquid).

The obtained pore water (pore water after 63 days from the start of the test) was subjected to qualitative analysis using a liquid chromatograph/time-of-flight mass spectrometer under the conditions in Table 1 below. That is, the pore water was subjected to component separation by liquid chromatography, and the separated pore water was subjected to component analysis by a time-of-flight mass spectrometer.

TABLE 1

| | |
|---|---|
| Measurement Instruments | LC part: Shimadzu Corporation LC-30AD |
| | MS part: Shimadzu Corporation LCMS-9030 |

LC Part Conditions

| | |
|---|---|
| Column | YMC YMC-Triart C18 |
| | (100 mm × 2.0 mm (id), 3 μm) |
| Column Temperature | 40° C. |
| Mobile Phase Flow Rate | 0.2 mL/min |
| Sample Injection Volume | 5 μL |
| Mobile Phase | A: 0.1% Formic acid/Water |
| | B: 0.1% Formic acid/Methanol |
| Gradient Conditions | 0→1 min A: 90% B: 10% |
| | 1→61 min A: 90→ 0% B: 10→ 100% |
| | 61→76 min A: 0% B: 100% |

MS Part Conditions

| | |
|---|---|
| Ionization Method | ESI-Negative |
| Mass Scan Range | m/z 50-2400 |

As a result, the monomers and oligomers corresponding to the columns described as A to J in Table 2 below were detected. In Table 2, x and y indicate the degree of polymerization of 3-hydroxybutyric acid (3-HB) and 3-hydroxyhexanoic acid (3-HH), respectively. For example, when x is 3 and x+y is 4, it indicates a tetramer in which three 3-hydroxybutyric acid units and one 3-hydroxyhexanoic acid unit are polymerized. During this detection, the presence or absence of peaks in the extracted ion chromatogram was confirmed under the condition of a mass error of +5 ppm for the monoisotopic exact mass of monovalent and divalent ions calculated from the molecular formulas of the degraded intermediates (oligomers with a total degree of polymerization of up to 20, composed of two types of monomers: 3-hydroxybutyric acid and 3-hydroxyhexanoic acid).

TABLE 2

Total degree of
polymerization (x + y)

| | | | | | |
|---|---|---|---|---|---|
| 1 | 2 | 3 | 4 | 5 | 6 |

| | | | | | | |
|---|---|---|---|---|---|---|
| Degree of | 0 | A | F | | | |
| polymerization | 1 | B | G | | | |
| of 3-HB (x) | 2 | | C | | | |
| | 3 | | | H | D | |
| | 4 | | | | E | |
| | 5 | | | | | I |
| | 6 | | | | | J |

Furthermore, in the obtained pore water (pore water after 63 days and 73 days from the start of the test), quantitative analysis of A to E among the degraded intermediates detected in Table 2 was performed using a liquid chromatograph/triple quadrupole mass spectrometer. The conditions are shown in Table 3 below.

TABLE 3

| | |
|---|---|
| Measurement Instruments | LC part: Shimadzu Corporation Nexera X2 |
| | MS part: Shimadzu Corporation LCMS-8060 |

LC Part Conditions

| | |
|---|---|
| Column | YMC YMC-Triart C18 |
| | (100 mm × 2.0 mm (id), 3 μm) |
| Column Temperature | 40° C. |
| Mobile Phase Flow Rate | 0.2 mL/min |
| Sample Injection Volume | 5 μL |
| Mobile Phase | A: 0.1% Formic acid/Water |
| | B: 0.1% Formic acid/Methanol |
| Gradient Conditions | 0→1 min A: 90% B: 10% |
| | 1→37 min A: 90→ 36% B: 10→ 64% |
| | 37→41 min A: 36→ 0% B: 64→ 100% |
| | 41→46 min A: 0% B: 100% |

MS Part Conditions

| Ionization Method | ESI-Negative (MRM) | |
|---|---|---|
| Monitor Ion | Quantifier Ion | Qualifier Ion |
| (m/z) | (Q1 → Q3) | (Q1 → Q3) |
| 3-HH | 131.2 → 58.9 | 131.2 → 40.9 |
| 3-HB | 103.1 → 59.0 | 103.1 → 40.9 |
| (2)H0B2^{※} | 189.1 → 83.1 | 189.1 → 189.1 |
| (4)H1B3^{※} | 389.2 → 203.1 | 389.2 → 83.1 |
| (4)H0B4^{※} | 361.2 → 189.1 | 361.2 → 83.1 |

^{※}(I)HmBn . . . I: Total degree of polymerization, m: Degree of polymerization of 3-HH, n: Degree of polymerization of 3-HB

In this quantitative analysis, the concentration was determined by collating the detected peak intensity for each degradation intermediate product with a calibration curve created by measuring standard products of 3-hydroxybutyric acid and 3-hydroxyhexanoic acid at multiple concentrations. The results are shown in Table 4 and FIG. 2. From these, it was found that in the degraded intermediates, the abundance of oligomers was greater than the abundance of monomers.

TABLE 4

| | Concentration in Sample [μg/mL] | | | | |
|---|---|---|---|---|---|
| Test Day | 3-HH | 3-HB | (2)H0B2 | (4)H1B3 | (4)H0B4 |
| Day 63 | 0.4 | 3.0 | 120 | 20 | 290 |
| Day 73 | 0.3 | 2.2 | 120 | 19 | 300 |

| | Concentration/MW = Molar Concentration [mol/mL] | | | | |
|---|---|---|---|---|---|
| Test Day | 3-HH | 3-HB | (2)H0B2 | (4)H1B3 | (4)H0B4 |
| Day 63 | 0.003 | 0.03 | 0.63 | 0.05 | 0.80 |
| Day 73 | 0.002 | 0.02 | 0.63 | 0.05 | 0.83 |

### Comparative Example 1

In Example 1, the supernatant seawater in the test bottles containing the marine biodegradable plastic pellets and test seawater was collected after a prescribed period (63 days, 73 days), and the supernatant seawater was analyzed under the same conditions as in Example 1 using a liquid chromatograph/time-of-flight mass spectrometer, but no degraded intermediates of the marine biodegradable plastic could be detected.

As is clear from Table 2 and Table 4, the monomer and oligomer components constituting the marine biodegradable plastic were detected in the pore water in an analyzable amount. From this, it was demonstrated that the components of the marine biodegradable plastic eluted through degradation of the marine biodegradable plastic pellets remain in the pore water contained in the biofilm, and that they can be detected. Therefore, it is understood that by implementing the analysis method of the first embodiment, information such as the degraded products of the marine biodegradable plastic and their amounts can be obtained.

1. A method for analyzing degraded intermediates of a marine biodegradable plastic, the method comprising:
an extraction step of extracting pore water present in a biofilm formed on a surface of the marine biodegradable plastic; and
a detection step of detecting the degraded intermediates in the pore water using mass spectrometry.

2. The analysis method according to claim 1, wherein in the detection step, a concentration of the degraded intermediates is detected.

3. The analysis method according to claim 1, wherein the degraded intermediates are a monomer and/or an oligomer constituting the marine biodegradable plastic.

4. The analysis method according to claim 3, wherein the degraded intermediates are a monomer and an oligomer constituting the marine biodegradable plastic.

5. The analysis method according to claim 3, wherein the oligomer is an oligomer with a total degree of polymerization of up to 20.

6. The analysis method according to claim 3, wherein the oligomer is an oligomer with a total degree of polymerization of up to 6.

7. The analysis method according to claim 3, wherein the oligomer is an oligomer with a total degree of polymerization of up to 4.

8. The analysis method according to claim 1, wherein in the extraction step, the pore water is extracted by filtering the biofilm formed on the surface of the marine biodegradable plastic.

9. The analysis method according to claim 1, wherein the mass spectrometry uses a time-of-flight mass spectrometer.

10. The analysis method according to claim 1, wherein in the detection step, after separating components of a solution in which the degraded intermediates have been extracted by liquid chromatography, mass spectrometry is performed.

11. The analysis method according to claim 10, wherein the liquid chromatography is performed by gradient elution.

12. The analysis method according to claim 1, wherein the marine biodegradable plastic has a structure represented by the following chemical formula. [chemical structure]
